(19) United States
(12) Patent Application Publication
(10) Pub. No.: US 2025/0329458 A1
(43) Pub. Date: Oct. 23, 2025

(54) SYSTEMS AND METHODS FOR PROVIDING A MEDICAL TESTING RECOMMENDATION

(71) Applicant: SPECTRUM MEDICAL DIAGNOSTICS, INC., Mississauga (CA)

(72) Inventor: Sharmila Sharmin SRIRAM HIRIYANNA, Mississauga (CA)

(21) Appl. No.: 19/075,055

(22) Filed: Mar. 10, 2025

Related U.S. Application Data
(60) Provisional application No. 63/636,173, filed on Apr. 19, 2024.

(51) Int. Cl. G16H 50/20; G16H 10/20; G16H 10/40; G16H 10/60; G16H 40/20; G16H 70/20

(52) U.S. Cl. CPC G16H 50/20; G16H 10/20; G16H 10/40; G16H 10/60; G16H 40/20; G16H 70/20

(57) ABSTRACT

A medical testing recommendation system and method are provided for identifying a plurality of point-of-care tests to be administered to a patient remotely from medical testing facilities. The medical testing recommendation system includes a point-of-care test database storing data related to the plurality of point-of-care tests available to be administered remotely from the testing facilities; and a processor in communication with the point-of-care test database. The processor is configured to: receive a patient data related to the patient, the patient data including a plurality of symptoms experienced by the patient and a patient personal data related to personal information and historical medical data of the patient; evaluate the patient data to identify a plurality of diseases associated with one or more symptoms of the plurality of symptoms; determine, with reference to the point-of-care test database, whether a point-of-care test is available for each disease of the plurality of diseases.

### CROSS-REFERENCE TO RELATED APPLICATION(S)

This application claims benefit of U.S. Provisional Patent Application No. 63/636,173 filed on Apr. 19, 2024. The content of U.S. Provisional Patent Application No. 63/636,173 is hereby incorporated by reference in its entirety.

### FIELD

The described embodiments relate to systems and methods for providing medical testing recommendations, and specifically, for identifying point-of-care tests to be administered to a patient.

### BACKGROUND

A point-of-care test (POCT) or a rapid diagnostic test (RDT) is a medical diagnostic test that can be administered to a patient at or near the point of care (e.g., at home). The POCT may not be required to be administered at a medical facility or a clinic. Additionally, no prior medical training may be required for administering the POCT. For example, a patient may self-administer the POCT. As another example, a care provider (e.g., a family member or a friend) may administer the POCT to the patient. The POCT can provide rapid diagnostic results (e.g., within minutes) thereby enabling rapid diagnosis of a patient's medical condition.

### SUMMARY

The various embodiments described herein generally relate to medical testing recommendation systems (and associated methods) for identifying a plurality of point-of-care tests to be administered to a patient.

In accordance with an embodiment, there is provided a medical testing recommendation system for identifying a plurality of point-of-care tests to be administered to a patient remotely from medical testing facilities. The medical testing recommendation system includes a point-of-care test database storing data related to the plurality of point-of-care tests available to be administered remotely from the testing facilities; and a processor in communication with the point-of-care test database. The processor is configured to: receive a patient data related to the patient, the patient data including a plurality of symptoms experienced by the patient and a patient personal data related to personal information and historical medical data of the patient; evaluate the patient data to identify a plurality of diseases associated with one or more symptoms of the plurality of symptoms; determine, with reference to the point-of-care test database, whether a point-of-care test is available for each disease of the plurality of diseases; in response to determining the point-of-care test is available for at least one disease of the plurality of diseases, evaluate the patient personal data to determine whether the patient is suitable for the point-of-care test and automatically acquire, via a network, the point-of-care test for the patient when the patient is determined suitable for the point-of-care test; receive, via a result interface, a test result from each point-of-care test administered to the patient; and evaluate the test result from each point-of-care test to offer a diagnostic recommendation for the patient.

In some embodiments, the processor is configured to receive a patient input in a natural-language format describing one or more symptoms experienced by the patient; and convert the one or more symptoms in the natural-language format into medical terminology.

In some embodiments, the processor is configured to determine the one or more symptoms in the natural-language format relates to a different number of medical terminologies.

In some embodiments, each point-of-care test can be administered by a non-medical professional remotely from medical testing facilities.

In some embodiments, the processor is configured to automatically initiate an electronic transaction, via the network, to order the point-of-care test for the patient when the patient is determined suitable for the point-of-care test.

In some embodiments, the processor is configured to determine a disease complexity level of the plurality of diseases identified from the patient data; and in response to determining the disease complexity level is within a remote test acceptance level, proceed to automatically acquire the point-of-care test, otherwise, instruct the patient to obtain direct medical care.

In some embodiments, the processor is configured to determine the disease complexity level exceeds the remote test acceptance level when a presence of two or more diseases of the plurality of diseases increases a medical risk for the patient based at least on the patient data.

In some embodiments, the processor is configured to apply a disease complexity machine-learning model to determine the disease complexity level of the plurality of diseases, the disease complexity machine-learning model being trained from a plurality of datasets relating medical risks associated with the plurality of diseases and patient data associated with a plurality of different patients.

In some embodiments, the processor is configured to assess an administration success likelihood based at least on the patient personal data and historical medical data, the administration success likelihood representing how likely the patient would properly administer the point-of-care test remotely from the medical testing facilities; and in response to determining the administration success likelihood is within a remote test acceptance level, proceed to automatically acquire the point-of-care test, otherwise, instruct the patient to obtain direct medical care.

In some embodiments, the processor is configured to apply an administration success machine-learning model to predict the administration success likelihood for the patient, the administration success machine-learning model being trained from a plurality of datasets relating test results generated from point-of-care tests and patient data associated with a plurality of different patients.

In some embodiments, the processor is configured to: determine the administration success likelihood is within the remote test acceptance level when the patient personal data indicates that an age of the patient is within a remote test age range.

In some embodiments, the processor is configured to: receive, via the result interface, an image of the administered point-of-care test; and apply an image analysis technique to the image for generating the test result.

In some embodiments, the processor is configured to instruct the patient to obtain direct medical care when the test result from one or more point-of-care tests indicates a high level of medical risk.

In accordance with an embodiment, there is provided a medical testing recommendation method for identifying a plurality of point-of-care tests to be administered to a patient remotely from medical testing facilities. The medical testing recommendation method includes: receiving, by a processor, a patient data related to the patient, the patient data including a plurality of symptoms experienced by the patient and a patient personal data related to personal information and historical medical data of the patient; evaluating, by the processor, the patient data to identify a plurality of diseases associated with one or more symptoms of the plurality of symptoms; determining, by the processor, with reference to stored data related to the plurality of point-of-care tests available to be administered remotely from the testing facilities, whether a point-of-care test is available for each disease of the plurality of diseases; in response to determining the point-of-care test is available for at least one disease of the plurality of diseases, evaluating, by the processor, the patient personal data to determine whether the patient is suitable for the point-of-care test and automatically acquiring, via a network, the point-of-care test for the patient when the patient is determined suitable for the point-of-care test; receiving, by the processor via a result interface, a test result from each point-of-care test administered to the patient; and evaluating the test result from each point-of-care test to offer a diagnostic recommendation for the patient.

In some embodiments, the method includes receiving, by the processor, a patient input in a natural-language format describing one or more symptoms experienced by the patient; and converting, by the processor, the one or more symptoms in the natural-language format into medical terminology.

In some embodiments, determining the one or more symptoms in the natural-language format relates to a different number of medical terminologies.

In some embodiments, each point-of-care test can be administered by a non-medical professional remotely from medical testing facilities.

In some embodiments, the method includes automatically initiating an electronic transaction, by the processor via the network, to order the point-of-care test for the patient when the patient is determined suitable for the point-of-care test.

In some embodiments, the method includes determining, by the processor, a disease complexity level of the plurality of diseases identified from the patient data; and in response to determining the disease complexity level is within a remote test acceptance level, proceeding to automatically acquire the point-of-care test, otherwise, instructing the patient to obtain direct medical care.

In some embodiments, the method includes determining, by the processor, the disease complexity level exceeds the remote test acceptance level when a presence of two or more diseases of the plurality of diseases increases a medical risk for the patient based at least on the patient data.

In some embodiments, the method includes applying, by the processor, a disease complexity machine-learning model to determine the disease complexity level of the plurality of diseases, the disease complexity machine-learning model being trained from a plurality of datasets relating medical risks associated with the plurality of diseases and patient data associated with a plurality of different patients.

In some embodiments, the method includes assessing, by the processor, an administration success likelihood based at least on the patient personal data and historical medical data, the administration success likelihood representing how likely the patient would properly administer the point-of-care test remotely from the medical testing facilities; and in response to determining the administration success likelihood is within a remote test acceptance level, proceed to automatically acquire the point-of-care test, otherwise, instruct the patient to obtain direct medical care.

In some embodiments, the method includes applying, by the processor, an administration success machine-learning model to predict the administration success likelihood for the patient, the administration success machine-learning model being trained from a plurality of datasets relating test results generated from point-of-care tests and patient data associated with a plurality of different patients.

In some embodiments, the method includes determining, by the processor, the administration success likelihood is within the remote test acceptance level when the patient personal data indicates that an age of the patient is within a remote test age range.

In some embodiments, the method includes receiving, by the processor via the result interface, an image of the administered point-of-care test; and applying an image analysis technique to the image for generating the test result.

In some embodiments, the method includes instructing the patient, by the processor, to obtain direct medical care when the test result from one or more point-of-care tests indicates a high level of medical risk.

### BRIEF DESCRIPTION OF THE DRAWINGS

Several embodiments will now be described in detail with reference to the drawings, in which:

FIG. 1 is a block diagram of components interacting with a medical testing recommendation system, in accordance with an example embodiment;

FIG. 2 is a flowchart of an example embodiment of various methods for identifying POCTs to be administered to a patient;

FIG. 3 is a screenshot of an example user interface provided by the system of FIG. 1 for receiving patient data, in accordance with an example embodiment;

FIG. 4 is a screenshot of an example user interface provided by the system of FIG. 1 showing the patient symptoms associated with an infectious disease condition, in accordance with an example embodiment;

FIG. 5 is a screenshot of an example user interface provided by the system of FIG. 1 showing patient questions and response corresponding to the patient symptoms of FIG. 4;

FIG. 6 is a screenshot of an example user interface showing a diagnostic recommendation provided by the system of FIG. 1; and

FIG. 7 is a screenshot of an example user interface showing a risk assessment graph provided by the system of FIG. 1, in accordance with an example embodiment.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to below as user devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

The disclosed systems and methods can identify a plurality of point-of-care tests (POCTs) to be administered to a patient remotely from medical testing facilities. This can enable rapid and efficient diagnosis of patient medical conditions without having the patient visit a medical testing facility. The disclosed systems and methods may be used as a pre-screening tool to enable more efficient usage of medical testing facilities.

The disclosed systems and methods can enable users of the system (e.g., patient or others) to provide patient data in natural language and convert the natural language input into medical terminology. This may provide improved access to POCTs to patients without medical training.

The disclosed systems and methods can evaluate patient data including patient symptoms to identify diseases associated with the symptoms. A disease risk score and/or a disease complexity level may be determined for the identified diseases. Further, the disclosed systems and methods can determine available POCTs for the identified diseases and an administration success likelihood of the POCT for the patient. This can improve overall efficiency of the testing recommendation system by reducing the data communication/storage resources and computational resources used in analyzing, communicating and/or storing test data from incorrectly administered tests.

The disclosed systems and methods can evaluate the test results from the POCTs to offer a diagnostic recommendation for the patient. The test results and/or diagnostic recommendations may be provided to users/patients in natural language to enable users/patients without medical training to take actions based on the test results/diagnostic recommendations.

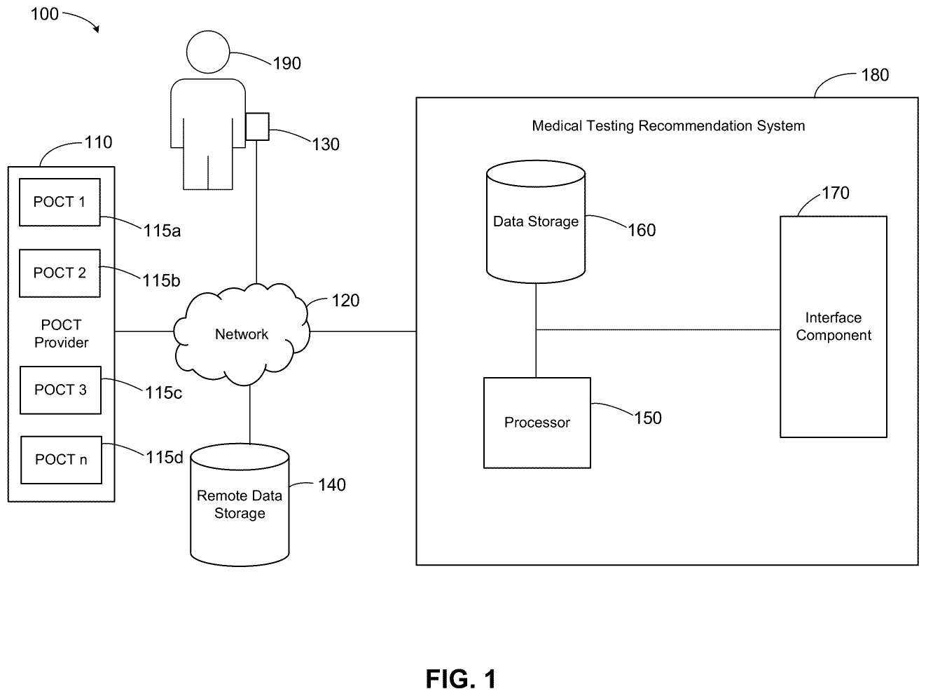

Reference is now made to FIG. 1, which illustrates a block diagram 100 of components interacting with a medical testing recommendation system 180 in accordance with an example embodiment. As shown in FIG. 1, system 180 can be in communication, via a network 120, with a POCT provider 110, a remote data storage 140, and a user device 130.

System 180 includes a processor 150, a data storage 160, and an interface component 170. Processor 150, data storage 160, and interface component 170 may be implemented in software or hardware, or a combination of software and hardware. Processor 150, data storage 160, and interface component 170 can be combined into a fewer number of components or may be separated into further components. System 180 may, in some embodiments, be split into multiple computing systems that may be distributed over a wide geographic area and connected via network 120.

Processor 150 is configured to control the operation of system 180. Processor 150 may be any suitable processors, controllers or digital signal processors that can provide sufficient processing power depending on the configuration, purposes and requirements of system 180. In some embodiments, processor 150 can include more than one processor with each processor being configured to perform different dedicated tasks. For example, processor 150 can identify one or more POCT to be administered to a patient.

Data storage 160 can include RAM, ROM, one or more hard drives, one or more flash drives or some other suitable data storage elements such as disk drives, etc. Data storage 160 may be used to store an operating system and programs as is commonly known by those skilled in the art. For instance, the operating system provides various basic operational processes for system 180. The programs may include various user programs so that a user/patient can interact with system 180 to perform various functions such as, but not limited to, providing input patient data and receiving medical testing recommendations.

Data storage 160 may include one or more databases. For example, data storage 160 can store a POCT database storing data related to the plurality of POCTs available to be administered.

In some embodiments, data storage 160 can store one or more machine learning models. The machine learning models may include, for example, a machine learning model for generating a POCT recommendation for a patient based on input data related to the patient. Further, data storage 160 may store training data used for training the one or more machine learning models.

In some embodiments, data storage 160 may store test results from POCTs administered to patients. Further, data storage 160 may store diagnostic recommendations provided for a patient. System 180 may use a combination of patient input data, test recommendations, test results and/or diagnostic recommendations to provide authorized users (e.g., physicians) with insights related to patient medical conditions.

Interface component 170 may be any interface that enables system 180 to communicate with other devices and systems. Interface component 170 may include at least one of an Internet, Local Area Network (LAN), Ethernet, Firewire, modem or digital subscriber line connection. Various combinations of these elements may be incorporated within interface component 170. For example, interface component 170 may receive input from various input devices, such as a mouse, a keyboard, a touchscreen, a thumbwheel a track-pad, a track-ball, a card-reader, voice recognition software and the like depending on the requirements and implementation of system 180.

Further, interface component 170 can provide a user interface (UI) for a user or a patient to interact with system 180. The user interface provided by interface component 170 can enable the user/patient to interact with system 180 in a number of ways, including but not limited to, providing input data related to the patient and receiving POCT recommendations.

User device 130 may be any networked device operable to connect to network 120. A networked device is a device capable of communicating with other devices through a network such as network 120. A networked device may couple to network 120 through a wired or wireless connection. These external user devices 130 may include at least a processor and a data storage, and may be an electronic tablet device, a personal computer, workstation, server, portable computer, mobile device, personal digital assistant, laptop, smart phone, WAP phone, and portable electronic devices or any combination of these. Although only one user device 130 is shown, it will be understood that more than one user device 130 can communicate with system 180 at any one time via network 120.

In the illustrated example, user device 130 is associated with a patient 190. Patient 190 can be any person experiencing one or more symptoms. Patient 190 may access system 180 to receive a medical testing recommendation. A connection request initiated from user device 130 may be initiated from a web browser or application and directed at the browser-based or application-based interface offered by interface component 170 of system 180.

In other examples, user device 130 may be associated with a medical facility (e.g., a pharmacy, a clinic, a laboratory, POCT provider 110, etc.). Patient 190 or an authorized user (e.g., a pharmacist, a clinician, a lab technician) may interact with system 180 using user device 130. For example, user device 130 may include a pharmacy kiosk accessible by patient 190.

POCT provider 110 may be any suitable medical facility (e.g., a pharmacy, a clinic, a laboratory, etc.) that provides POCTs. For example, POCT provider 110 may include multiple POCTs 115a-115d. Although only one POCT provider 110 is shown for ease of exposition, it will be understood that more than one POCT provider 110 can communicate with system 180 at any one time.

In some examples, patient 190 may be located remotely from POCT provider 110. POCT provider 110 may use any suitable method to provide one or more POCTs to patient 190 (e.g., via mail delivery).

In other examples, patient 190 may be located at POCT provider 110. For example, patient 190 may be located at a pharmacy and receive the one or more POCTs directly in person.

POCTs 115 can be any suitable POCT or RDT that may be administered to patient 190 to diagnose a medical condition. POCTs 115 may be operable using any suitable specimen, for example, urine, oral fluid, nasal/nasopharyngeal, whole blood etc. POCTs 115 may be used for different types of testing including, for example, routine bio monitoring (e.g., hemoglobin A1C, vitamin D, thyroid stimulating hormone (TSH), cholesterol, prostate-specific antigen (PSA), ferritin), substance abuse testing (e.g., fentanyl, cannabis, cocaine), cardiac panels (e.g., C-reactive protein (CRP), troponin), infectious disease testing (e.g., Covid, Strep, Influenza A/B, respiratory syncytial virus (RSV), Lyme disease, mononucleosis), women's health testing (e.g., ovulation, human chorionic gonadotropin (hCG) pregnancy, Actim® PROM, Actim Partus), allergen testing, etc.

Remote data storage 140 can include RAM, ROM, one or more hard drives, one or more flash drives or some other suitable data storage elements. Remote data storage 140 may also include one or more database(s) or file system(s). Although only one remote data storage 140 is shown for ease of exposition, there may be multiple remote data storage 140 distributed over a wide geographic area and connected via network 120. In some embodiments, remote data storage 140 may store historical medical data of patients. System 180 may access the stored historical medical data of a patient to identify one or more POCTs to be administered to the patient. In some embodiments, remote data storage 140 can be used as a back-up storage to data storage 160, for example, to provide data storage redundancy. In some embodiments, data storage 160 may store data that is frequently accessed by processor 150 and remote data storage 140 may store data less frequently accessed by processor 150.

Network 120 may be any network capable of carrying data including the Internet, Ethernet, plain old telephone service (PTOS) line, public switch telephone network (PSTN), integrated services digital network (ISDN), digital subscriber line (DSL), coaxial cable, fiber optics, satellite, mobile, wireless (e.g., Wi-Fi, WiMAX), SS7 signaling network, fixed line, local area network, wide area network, and others, including any combination of these, capable of interfacing with, and enabling communication between system 180, user device 130, POCT provider 110 and remote data storage 140.

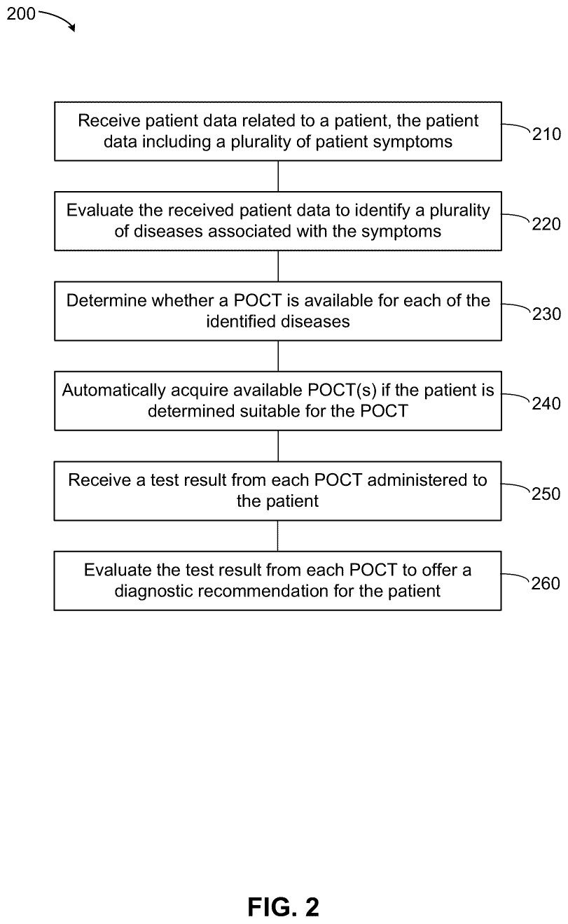

Reference is now made to FIG. 2, showing a flowchart of an example method 200 for identifying POCTs to be administered to a patient. Method 200 may be executed using any suitable system. For example, method 200 may be executed using system 180 shown in FIG. 1 and reference will be made simultaneously to components shown in FIG. 1.

In some examples, system 180 may execute method 200 for a new patient. No historical medical data may be available for the new patient. In other examples, system 180 may execute method 200 for a returning patient. Historical medical data may be available for the returning patient. For example, data storage 160 and/or remote data storage 140 may store historical medical data in a patient record that is associated with the returning patient.

System 180 may execute method 200 at various times. For example, system 180 may execute method 200 in response to a user input. The user input may be received from user device 130. For example, user device 130 may be associated with patient 190 and the user input may include patient data provided by patient 190. As another example, user device 130 may be associated with a medical facility and the user input may include a user command from a user (e.g., a pharmacist, a clinician, a laboratory technician, etc.) at the medical facility. In some examples, system 180 may execute method 200 continuously or periodically.

At 210, processor 150 receives patient data related to the patient (e.g., patient 190). The received patient data may include patient personal data and one or more symptoms experienced by the patient.

Processor 150 may receive the patient data from data storage 160, remote data storage 140 and/or user device 130. For example, processor 150 may receive at least a portion of the patient data from data storage 160 and/or remote data storage 140 for a returning patient. Processor 150 may retrieve the patient data from a patient record associated with the returning patient. In some examples, processor 150 may receive all of the patient data from user device 130. For example, a new patient may provide all of the patient data via user device 130.

The patient personal data may include data related to personal information and historical medical data of the patient. For example, the personal information may include name, age, gender, ethnicity, height, weight, and/or body mass index (BMI) information of the patient. The historical medical data may include historical medical information of the patient including, for example, symptoms, test results, diagnoses, prescribed medications and/or drug usage. For a returning patient, the historical medical data may include test results from one or more POCTs previously recommended by system 180 to the patient. In some embodiments, the patient personal data may include data related to the patient's family medical history, exercise habits, diet, and/or sunshine exposure.

In some embodiments, processor 150 may store the received patient data in a patient record in data storage 160 and/or remote data storage 140 (subject to applicable medical data storage laws/regulations). Processor 150 may use the stored patient data to identify one or more POCTs to be administered to the patient. In some embodiments, processor 150 may use the stored patient data to generate risk assessments and/or insights based on aggregated patient data for multiple patients. Processor 150 may provide the generated risk assessments and/or insights to an authorized user (e.g., a medical practitioner) subject to applicable medical data privacy laws/regulations.

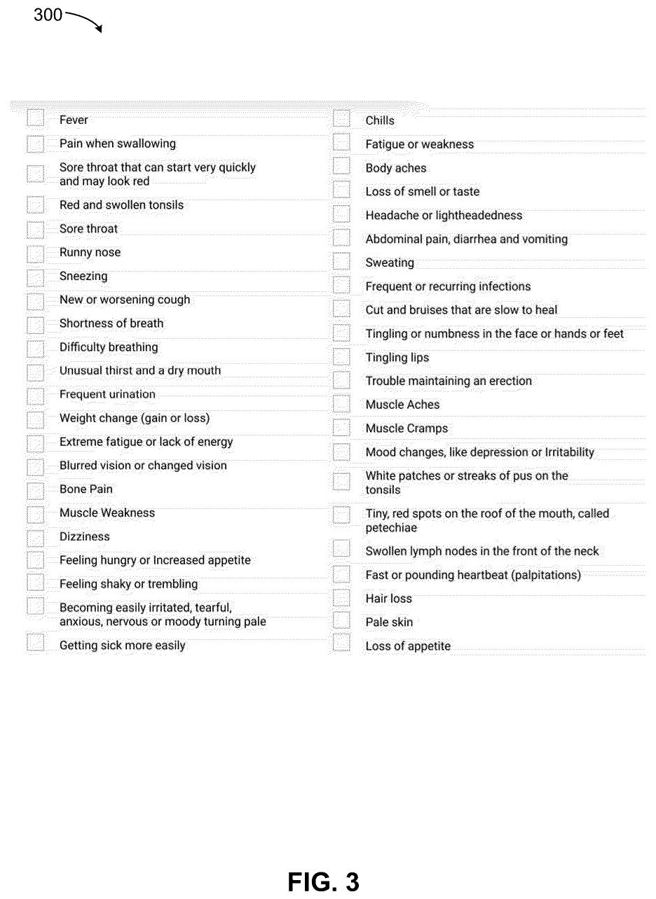

As described above, the received patient data may include data for one or more symptoms experienced by the patient. Processor 150 may use any suitable method to receive the patient symptoms data. For example, system 180 may present a list of symptoms via a user interface on user device 130. Reference is now additionally made to FIG. 3. FIG. 3 is screenshot 300 of an example user interface provided by system 180. A user (e.g., patient 190) may select checkboxes corresponding to the experienced symptoms to provide the corresponding data to processor 150.

In some embodiments, processor 150 may receive the patient input in a natural-language format describing one or more symptoms experienced by the patient. For example, system 180 may present a text input box or a voice input prompt to a user (e.g., patient 190) via a user interface on user device 130.

Processor 150 may perform natural language processing of the received patient input to convert one or more symptoms in the natural-language format into medical terminology. In some examples, the number of symptoms in the natural-language format may relate to the same number of medical terminologies. For example, processor 150 may convert a “shivering” symptom in the natural-language format into a “hypothermia” medical terminology. In some examples, the number of symptoms in the natural-language format may relate to a different number of medical terminologies. For example, processor 150 may convert “loss of appetite” and “weight loss” symptoms in the natural-language format into an “anorexia” medical terminology.

At 220, processor 150 evaluates the received patient data to identify one or more diseases associated with the symptom(s) experienced by the patient (symptom(s) included in the patient data received at 210). The term “disease” as used herein may refer to any medical condition being experienced by the patient.

In some embodiments, processor 150 may evaluate the received patient data to identify diseases by inputting the received patient data into a trained machine learning model. Processor 150 may train the machine learning model using training data that includes historical patient data, corresponding test recommendations by medical practitioners and the corresponding test results. The training data may include data corresponding to different medical conditions, patients, medical practitioners, and tests. A larger size and/or variety of training data may enable training of higher accuracy machine learning models. The training data may include data to train the machine learning model to detect cross-reactivity issues. For example, one or more patient symptoms may be associated with the side-effects of a medication instead of a disease condition. As another example, one or more patient medications may interfere with the results of an administered test. Using a machine learning model to evaluate the received patient data may provide higher accuracy compared with alternative approaches.

In other embodiments, processor 150 may not use a machine learning model to evaluate the received patient data to identify diseases associated with the patient symptoms. For example, processor 150 may use a set of mathematical functions and/or a decision tree including conditional control statements to evaluate the received patient data. This may require a lower amount of computational resources compared with the computational and data resources required for training and using machine learning models.

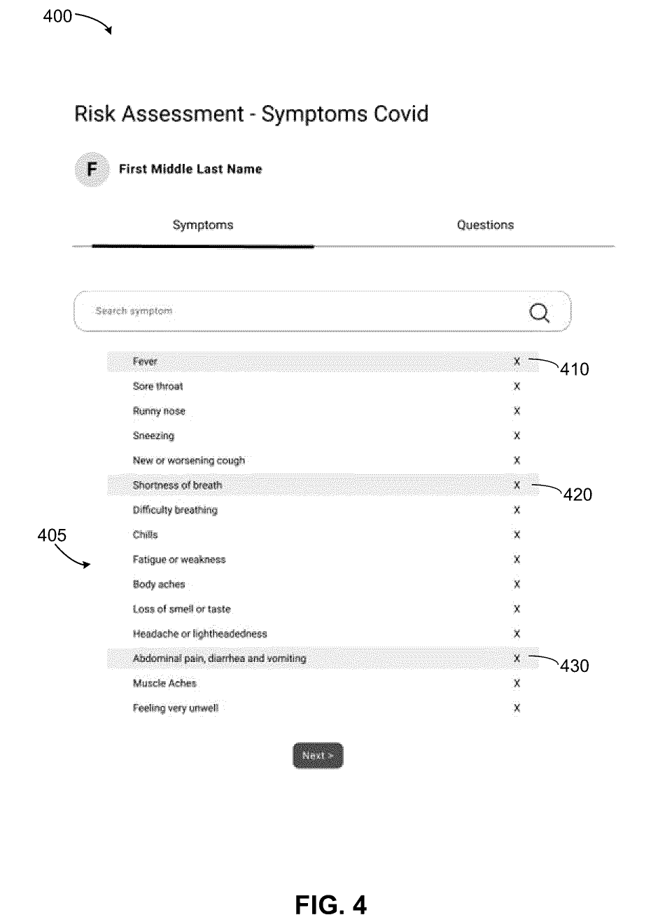

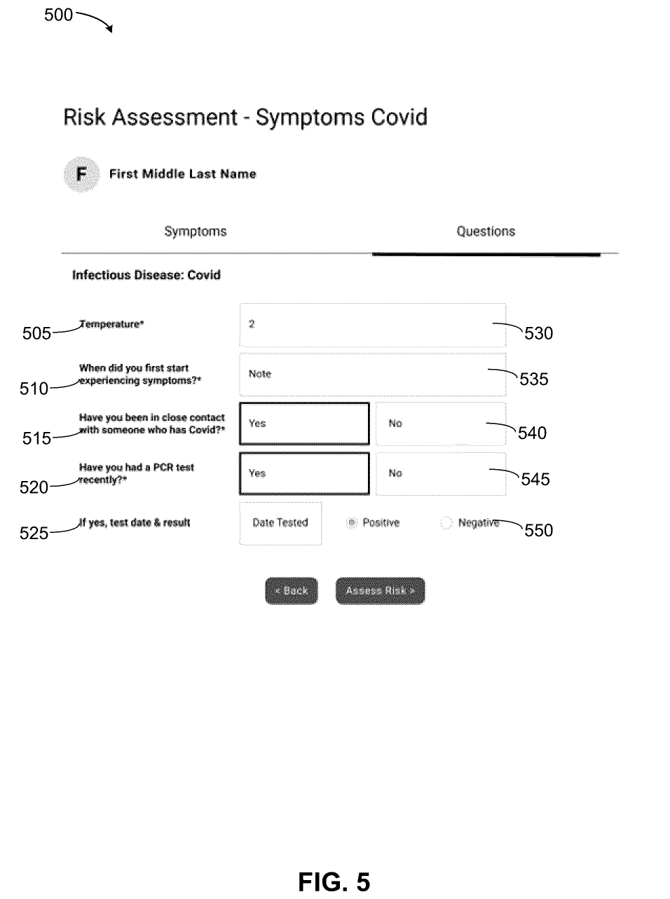

In some embodiments, processor 150 may generate one or more patient questions based on the symptoms experienced by the patient. Reference is now additionally made to FIGS. 4 and 5. FIG. 4 is a screenshot 400 of an example user interface provided by system 180 showing the symptoms 405 associated with an infectious disease condition (Covid) and the symptoms 410, 420 and 430 experienced by the patient. FIG. 5 is screenshot 500 of an example user interface provided by system 180 showing patient questions 505, 510, 515, 520 and 525 and patient responses 530, 535, 540, 545 and 550.

Processor 150 may use any suitable method to generate the patient questions. For example, as described herein above, one or more trained machine learning models may be used to evaluate the patient data. The training data used for training the machine learning models may be augmented using historical patient questions asked by medical practitioners in response to specific patient symptoms. The machine learning models trained using the augmented training data may generate the patient questions in response to the input patient data.

System 180 may use interface component 170 to present the generated patient questions to a user (e.g., patient 190). The generated patient questions may be presented to the user via a user interface on user device 130. The user may provide patient responses 530-550 to system 180 via user device 130.

Processor 150 may further input the received patient responses into the trained machine learning models. The trained machine learning models may identify one or more diseases associated with the symptoms experienced by the patient based on the patient data received at 210 in combination with the patient responses 530-550 to patient questions 505-525. Using the augmented training data for training the machine learning model and using the additional data provided by the patient responses may improve the accuracy of the machine learning models in identifying diseases associated with the patient symptoms.

In some embodiments, processor 150 may determine a risk score associated with each of the identified diseases. Processor 150 may use any suitable method to determine the risk score. For example, the machine learning models may be trained to determine a risk score for each of the identified diseases. As another example, processor 150 may use a risk score function to determine a risk score for each of the identified diseases.

The risk score may be based on one or more factors including for example, a number of symptoms experienced by the patient that are associated with the disease, a severity of the symptoms, patient personal information (e.g., age, gender, ethnicity, etc.) and/or patient medical history (e.g., previous diagnoses, pre-existing medical conditions, diagnostic test data, etc.).

In some embodiments, processor 150 may generate an output notification if the risk score associated with any of the identified diseases exceeds a threshold level.

System 180 may use interface component 170 to present the generated output notification to the user (e.g., via a user interface on user device 130). The output notification may instruct the patient to seek immediate medical attention for the identified disease.

In some embodiments, processor 150 may store risk scores determined at 220 in a patient record. For example, the patient record may be stored in data storage 160 and/or remote data storage 140.

In some embodiments, processor 150 may determine a disease complexity level of a plurality of diseases identified at 220. The disease complexity level may indicate that the presence of two or more diseases of the plurality of identified diseases increases a medical risk for the patient based at least on the patient data. For example, the disease complexity level may indicate an increased medical risk based on the determined risk scores for the identified diseases, and age, medical history, prescribed medications for patient 190.

Processor 150 may use any suitable method to determine the disease complexity level. For example, processor 150 may input the plurality of identified diseases and the patient data into a disease complexity machine learning model trained to output the disease complexity level. The disease complexity machine learning model may be trained using training data including datasets relating medical risks associated with different combinations of the plurality of diseases and patient data associated with different patients. A larger size and/or variety of training data may enable training of higher accuracy disease complexity machine learning models.

Processor 150 may compare the determined disease complexity level with a remote test acceptance level. If the determined disease complexity level is within the remote test acceptance level, processor 150 may proceed to determine whether a POCT is available for each of the identified diseases (at 230, as described herein below) and to automatically acquire the POCT (at 240, as described herein below).

If the determined disease complexity level exceeds the remote test acceptance level, processor 150 may generate an output notification. System 180 may use interface component 170 to present the generated output notification to the user (e.g., via a user interface on user device 130). The output notification may instruct the patient to obtain direct/immediate medical care.

In some embodiments, processor 150 may store disease complexity levels determined at 220 in a patient record. For example, the patient record may be stored in data storage 160 and/or remote data storage 140.

At 230, processor 150 determines whether a POCT is available for each disease identified at 220. Processor 150 may determine available POCTs with reference to a POCT database. The POCT database may be stored, for example, in data storage 160 and/or remote data storage 140. The POCT database may store data indicating the suitability of different POCTs to diagnose one or more of the identified diseases.

Each POCT may be administered by a non-medical professional remotely from medical testing facilities. For example, a patient may self-administer the POCT. The POCT may be administered without requiring the patient to visit a medical facility (e.g., a pharmacy, a clinic, a laboratory). For example, the POCT may be mailed to the patient from POCT provider 110. In some examples, the patient may visit a medical facility (e.g., a pharmacy, a clinic, a laboratory) to acquire the POCT and the POCT may be administered at the medical facility.

At 240, processor 150 evaluates the patient personal data to determine whether the patient is suitable for one or more available POCTs determined at 230. In some embodiments, processor 150 may evaluate the suitability of the patient by assessing an administration success likelihood of the POCT for the patient. The administration success likelihood can represent how likely the patient would properly administer the POCT remotely from the medical testing facilities.

The administration success likelihood may be based at least on the patient personal data and historical medical data. For example, the administration success likelihood may be determined to be within a remote test acceptance level when the patient personal data indicates that an age of the patient is within a remote test age range. As another example, the administration success likelihood may be determined to be within a remote test acceptance level when the historical medical data indicates previous successful remote administration of one or more POCTs.

Processor 150 may use any suitable method to assess the administration success likelihood. For example, processor 150 may input the patient personal data and historical medical data into an administration success machine learning model trained to output an administration success likelihood prediction. The administration success machine learning model may be trained using training data including datasets relating test results generated from POCTs and patient data associated with different patients. A larger size and/or variety of training data may enable training of higher accuracy administration success machine learning models.

Processor 150 may determine that the patient is suitable for the available POCT when the assessed administration success likelihood is with the remote test acceptance level. In response, processor 150 may proceed to automatically acquire the POCT from POCT provider 110. For example, processor 150 may automatically initiate an electronic transaction, via network 120, to order the POCT for the patient.

If the assessed administration success likelihood exceeds the remote test acceptance level, processor 150 may provide a corresponding output notification. System 180 may use interface component 170 to present the generated output notification to the user (e.g., via a user interface on user device 130). The output notification may instruct the patient to obtain direct medical care. For example, the output notification may instruct the patient to visit a medical facility (e.g., a hospital, a pharmacy, a clinic, a laboratory) to consult a medical practitioner and/or to undergo one or more diagnostic tests.

Method 200 may improve the efficiency and/or performance of system 180 by determining the administration success likelihood of the available POCTs. For example, this may improve the system efficiency by reducing the computational resources used in analyzing test data from incorrectly administered tests. As another example, this may improve the system efficiency by reducing the data communication and/or data storage resources used for test data from incorrectly administered tests.

Method 200 may proceed directly from 230 to 250 in cases where the patient visits a medical facility to acquire and administer the POCT.

At 250, processor 150 receives a test result from each POCT administered to the patient. System 180 may use interface component 170 to present a result interface on user device 130. The result interface may be operable to receive the test results in different data formats.

In some embodiments, processor 150 may receive an image of the administered POCT. For example, patient 190 may self-administer a POCT and upload an image of the test result via the result interface. Processor 150 may receive the uploaded image and apply any suitable image analysis technique to the image for generating the test result. For example, the POCT may be a Covid-19 rapid antigen test and processor 150 may analyze the image to detect presence of the line indicator for a positive test. As another example, the POCT may be a Vitamin-D test and processor 150 may analyze the image to generate the test result based on an intensity of the line indicators.

In some embodiments, processor 150 may receive the test results in text format. For example, a user (e.g., patient 190) may upload a text file containing the test results via the result interface. As another example, a user may enter test results via a text box provided by the result interface.

At 260, processor 150 evaluates the test result from each POCT to offer a diagnostic recommendation for the patient. Processor 150 may update the risk scores associated with each of the identified diseases based on the test result from each POCT.

Processor 150 may generate a diagnostic recommendation based on the updated risk scores. For example, processor 150 may generate an output notification instructing the patient to obtain direct medical care for an identified disease when the corresponding risk score indicates a high level of medical risk. As another example, processor 150 may generate an output notification recommending a clinical/laboratory test based on a moderate risk score and an inconclusive POCT result.

In some examples, user device 130 may be associated with a medical facility (e.g., a pharmacy, clinic, laboratory). Method 200 may be executed based on inputs provided (via user device 130) by a user other than patient 190 (e.g., a pharmacist, a lab technician). Processor 150 may provide the diagnostic recommendation to the user. The user may take a different action compared with the diagnostic recommendation. The actual user action may be provided as an input to system 180. System 180 may update the training data based on the actual user action. The updated training data may be used to retrain one or more machine learning models used by method 200. The retraining may further improve the accuracy of the machine learning models.

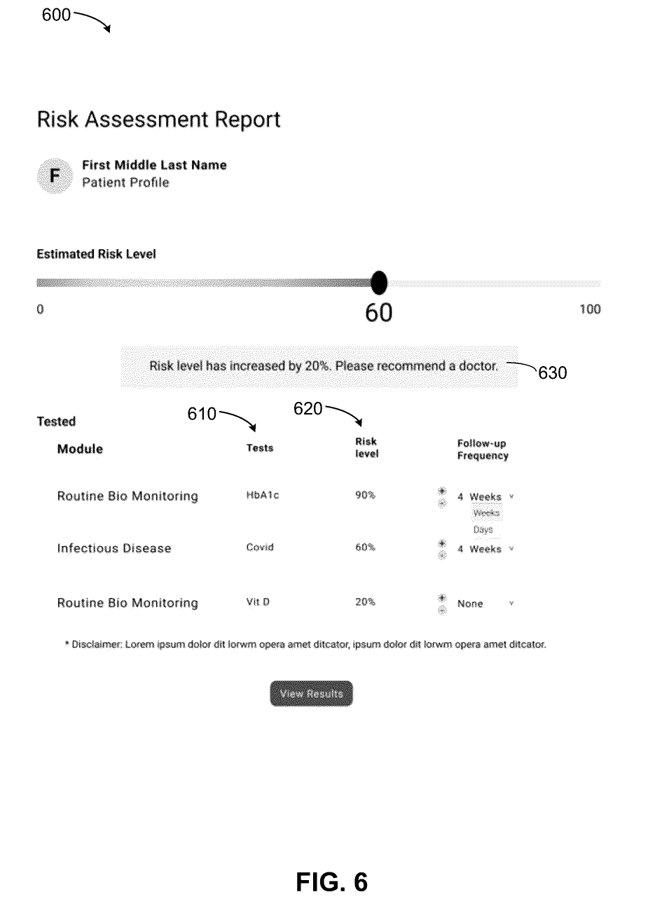

Reference is now additionally made to FIG. 6. FIG. 6 is a screenshot 600 of an example user interface showing a diagnostic recommendation 630 provided by system 180. The diagnostic recommendation 630 may be based on POCTs 610 administered to patient 190. The risk levels 620 may be based on the POCT results and patient data related to patient 190.

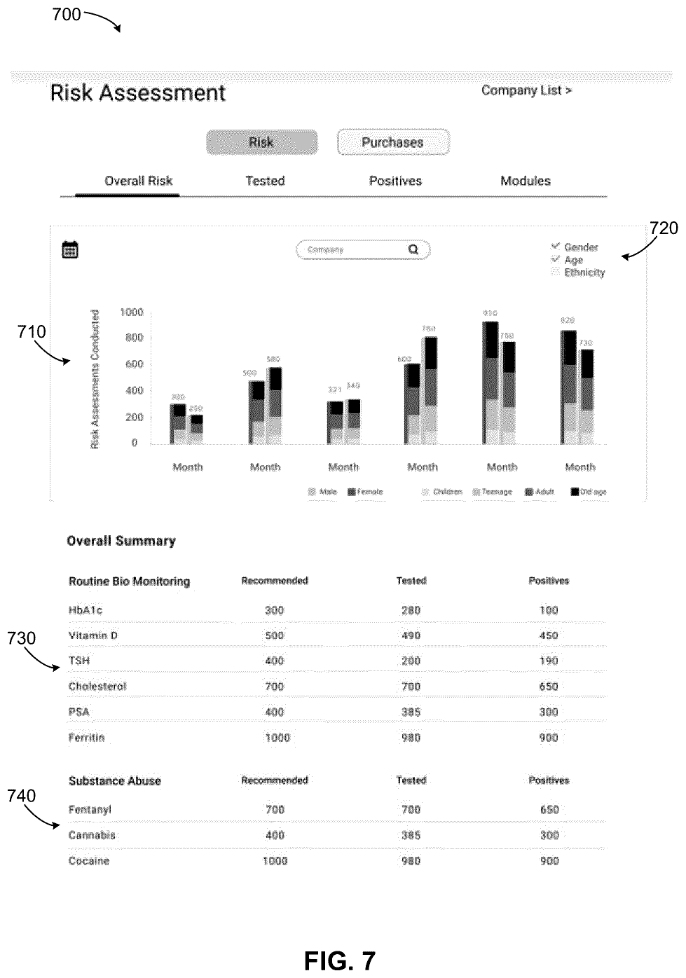

In some embodiments, processor 150 may provide one or more medical testing insights to a user. The user may be, for example, a medical practitioner. The insight may be based on aggregated data for multiple patients and tests. Reference is now additionally made to FIG. 7. FIG. 7 is a screenshot 700 of an example user interface showing a risk assessment graph 710. Processor 150 may enable a user to view risk assessment graph corresponding to different combinations of patient variables 720. In the illustrated example, graph 710 corresponds to patient variables 720 of “Gender” and “Age”. Screenshot 700 may also include portions 730 and 740 showing a summary of the underlying test data used to generate graph 710. The test data may be grouped into modules based on the type of tests/diagnosed medical conditions.

In some embodiments, processor 150 may provide the medical testing insights using one or more machine learning models. The machine learning models may be training to generate insights/predictions based on historical test and patient data.

Various embodiments have been described herein by way of example only. Various modification and variations may be made to these example embodiments without departing from the spirit and scope of the invention, which is limited only by the appended claims. Also, in the various user interfaces illustrated in the drawings, it will be understood that the illustrated user interface text and controls are provided as examples only and are not meant to be limiting. Other suitable user interface elements may be possible.

We claim:

1. A medical testing recommendation system for identifying a plurality of point-of-care tests to be administered to a patient remotely from medical testing facilities, the medical testing recommendation system comprising:
a point-of-care test database storing data related to the plurality of point-of-care tests available to be administered remotely from the testing facilities; and
a processor in communication with the point-of-care test database, the processor configured to:
receive a patient data related to the patient, the patient data comprising a plurality of symptoms experienced by the patient and a patient personal data related to personal information and historical medical data of the patient;
evaluate the patient data to identify a plurality of diseases associated with one or more symptoms of the plurality of symptoms;
determine, with reference to the point-of-care test database, whether a point-of-care test is available for each disease of the plurality of diseases;
in response to determining the point-of-care test is available for at least one disease of the plurality of diseases, evaluate the patient personal data to determine whether the patient is suitable for the point-of-care test and automatically acquire, via a network, the point-of-care test for the patient when the patient is determined suitable for the point-of-care test;
receive, via a result interface, a test result from each point-of-care test administered to the patient; and
evaluate the test result from each point-of-care test to offer a diagnostic recommendation for the patient.

2. The medical testing recommendation system of claim 1, wherein the processor is configured to:
receive a patient input in a natural-language format describing one or more symptoms experienced by the patient; and
convert the one or more symptoms in the natural-language format into medical terminology.

3. The medical testing recommendation system of claim 2, wherein the processor is configured to:
determine the one or more symptoms in the natural-language format relates to a different number of medical terminologies.

4. The medical testing recommendation system of claim 1, wherein the processor is configured to:
determine a disease complexity level of the plurality of diseases identified from the patient data; and
in response to determining the disease complexity level is within a remote test acceptance level, proceed to automatically acquire the point-of-care test, otherwise, instruct the patient to obtain direct medical care.

5. The medical testing recommendation system of claim 4, wherein the processor is configured to:
determine the disease complexity level exceeds the remote test acceptance level when a presence of two or more diseases of the plurality of diseases increases a medical risk for the patient based at least on the patient data.

6. The medical testing recommendation system of claim 4, wherein the processor is configured to:
apply a disease complexity machine-learning model to determine the disease complexity level of the plurality of diseases, the disease complexity machine-learning model being trained from a plurality of datasets relating medical risks associated with the plurality of diseases and patient data associated with a plurality of different patients.

7. The medical testing recommendation system of claim 1, wherein the processor is configured to:
assess an administration success likelihood based at least on the patient personal data and historical medical data, the administration success likelihood representing how likely the patient would properly administer the point-of-care test remotely from the medical testing facilities; and
in response to determining the administration success likelihood is within a remote test acceptance level, proceed to automatically acquire the point-of-care test, otherwise, instruct the patient to obtain direct medical care.

8. The medical testing recommendation system of claim 7, wherein the processor is configured to:
apply an administration success machine-learning model to predict the administration success likelihood for the patient, the administration success machine-learning model being trained from a plurality of datasets relating test results generated from point-of-care tests and patient data associated with a plurality of different patients.

9. The medical testing recommendation system of claim 7, wherein the processor is configured to:
determine the administration success likelihood is within the remote test acceptance level when the patient personal data indicates that an age of the patient is within a remote test age range.

10. The medical testing recommendation system of claim 1, wherein the processor is configured to:
receive, via the result interface, an image of the administered point-of-care test; and
apply an image analysis technique to the image for generating the test result.

11. A medical testing recommendation method for identifying a plurality of point-of-care tests to be administered to a patient remotely from medical testing facilities, the medical testing recommendation method comprising:
receiving, by a processor, a patient data related to the patient, the patient data comprising a plurality of symptoms experienced by the patient and a patient personal data related to personal information and historical medical data of the patient;
evaluating, by the processor, the patient data to identify a plurality of diseases associated with one or more symptoms of the plurality of symptoms;
determining, by the processor, with reference to stored data related to the plurality of point-of-care tests available to be administered remotely from the testing facilities, whether a point-of-care test is available for each disease of the plurality of diseases;
in response to determining the point-of-care test is available for at least one disease of the plurality of diseases, evaluating, by the processor, the patient personal data to determine whether the patient is suitable for the point-of-care test and automatically acquiring, via a network, the point-of-care test for the patient when the patient is determined suitable for the point-of-care test;
receiving, by the processor via a result interface, a test result from each point-of-care test administered to the patient; and
evaluating the test result from each point-of-care test to offer a diagnostic recommendation for the patient.

12. The medical testing recommendation method of claim Error! Reference source not found.1, wherein the method comprises:
receiving, by the processor, a patient input in a natural-language format describing one or more symptoms experienced by the patient; and
converting, by the processor, the one or more symptoms in the natural-language format into medical terminology.

13. The medical testing recommendation method of claim 12, wherein:
determining the one or more symptoms in the natural-language format relates to a different number of medical terminologies.

14. The medical testing recommendation method of claim 11, wherein the method comprises:
determining, by the processor, a disease complexity level of the plurality of diseases identified from the patient data; and
in response to determining the disease complexity level is within a remote test acceptance level, proceeding to automatically acquire the point-of-care test, otherwise, instructing the patient to obtain direct medical care.

15. The medical testing recommendation method of claim 14, wherein the method comprises:
determining, by the processor, the disease complexity level exceeds the remote test acceptance level when a presence of two or more diseases of the plurality of diseases increases a medical risk for the patient based at least on the patient data.

16. The medical testing recommendation method of claim 14, wherein the method comprises:
applying, by the processor, a disease complexity machine-learning model to determine the disease complexity level of the plurality of diseases, the disease complexity machine-learning model being trained from a plurality of datasets relating medical risks associated with the plurality of diseases and patient data associated with a plurality of different patients.

17. The medical testing recommendation method of claim 11, wherein the method comprises:
assessing, by the processor, an administration success likelihood based at least on the patient personal data and historical medical data, the administration success likelihood representing how likely the patient would properly administer the point-of-care test remotely from the medical testing facilities; and
in response to determining the administration success likelihood is within a remote test acceptance level, proceed to automatically acquire the point-of-care test, otherwise, instruct the patient to obtain direct medical care.

18. The medical testing recommendation method of claim 17, wherein the method comprises:
applying, by the processor, an administration success machine-learning model to predict the administration success likelihood for the patient, the administration success machine-learning model being trained from a plurality of datasets relating test results generated from point-of-care tests and patient data associated with a plurality of different patients.

19. The medical testing recommendation method of claim 17, wherein the method comprises:
determining, by the processor, the administration success likelihood is within the remote test acceptance level when the patient personal data indicates that an age of the patient is within a remote test age range.

20. The medical testing recommendation method of claim 11, wherein the method comprises:
receiving, by the processor via the result interface, an image of the administered point-of-care test; and
applying an image analysis technique to the image for generating the test result.
